(19) United States
(12) Patent Application Publication
(10) Pub. No.: US 2024/0148949 A1
(43) Pub. Date: May 9, 2024

(54) Access System Including Flexible Needle

(71) Applicant: Bard Peripheral Vascular, Inc., Franklin Lakes, NJ (US)

(72) Inventors: Adam T. Martin, Holly Springs, NC (US); Nicholas J. Jardine, Holly Springs, NC (US)

(21) Appl. No.: 18/281,377

(22) PCT Filed: Mar. 10, 2022
(86) PCT No.: PCT/US22/19821

§ 371 Date: Sep. 11, 2023

Related U.S. Application Data
(60) Provisional application No. 63/162,406, filed on Mar. 17, 2021.

(51) Int. Cl. A61M 1/36; A61M 25/06; A61M 39/02

(52) U.S. Cl. CPC A61M 1/3661; A61M 25/0606; A61M 39/0208; A61M 2205/02

(57) ABSTRACT

Embodiments disclosed herein are directed to an access system including a flexible needle configured to negotiate a non-linear pathway to access a subcutaneous port. Optionally, the access system can include a needle guide to facilitate alignment of the needle with the subcutaneous port. The needle can be configured to be flexible enough to have some bending, or deflection capability but also possess sufficient columnar strength to prevent buckling when piercing the skin and entering the port. The flexible needle can be deflected from a central axis to overcome any misalignment with the receiving cup and/or conduit of the port. Once within the port conduit the needle can negotiate the non-linear, or tortuous port conduit pathway, and stream-line the insertion procedure.

### PRIORITY

This application claims the benefit of priority to U.S. Provisional Application No. 63/162,406, filed Mar. 17, 2021, which is incorporated by reference in its entirety into this application.

### SUMMARY

Briefly summarized, embodiments disclosed herein are directed to an access system including a flexible needle for accessing a port, or similar subcutaneous medical device, for example a hemodialysis port for use in a home environment. Optionally, the access system can further include a needle guide or similar device disposed on a skin surface and configured to align the flexible needle with the port.

Dialysis patients typically travel to dialysis treatment centers to receive their treatment. This can occur multiple times per week (e.g. between three to seven times per week) and typically require the patient to remain on site for several hours at a time. Some patients however have the option to perform the dialysis therapy from the comfort of their home. Conventional access to subcutaneous access ports require a rigid needle, with a soft cannula disposed thereon. The user pierces the skin with the needle and creates a pathway to the port opening. The user can advance the needle until the needle tip impinges the inner surface of the port conduit. The needle must then be retracted slightly, termed “backing off”, to allow the soft cannula to pass over the needle and flex through the angled conduit path, and optionally through the port valving mechanism.

A major concern with accessing a subcutaneous dialysis port is palpating and locating the port orifice or receiving cup. When using a rigid needle, a user must align the needle with the conduit, within a narrow window or “angle of attack” to ensure a straight pathway into the port. The user must then perform the complex process of “backing off” the needle to urge the soft cannula over the rigid needle tip, through the conduit and through the valve mechanism without kinking or collapsing the cannula. Should the user impinge and retract the needle too far, the soft cannula does not have enough internal support from the needle, and can result in the cannula buckling when attempting to pass through the port or valve structures. Alternatively, the user can “back off” too far resulting in disengaging the port altogether. These problems are further complicated when the patient is carrying out the access event by themselves in a home setting, and must perform the palpation and insertion with one hand and/or at an inconvenient angle. As a result, there is an increased risk of mis-sticks and reattempted access events, leading to an increase in scar tissue or tissue degradation at the access site. This can be of particular importance for dialysis patients who require repeated access events with little heal-time in between.

Embodiments disclosed herein are directed to an access system including a flexible needle configured to negotiate a non-linear pathway to access the subcutaneous port. Optionally, the access system can include a needle guide to facilitate alignment of the needle with the subcutaneous port. The needle can be configured to be flexible enough to have some bending, or deflection capability but also possess sufficient columnar strength to prevent buckling when piercing the skin and entering the port. The flexible needle can be deflected from a central axis to overcome any misalignment with the receiving cup and/or conduit of the port. Once within the port conduit the needle can negotiate the non-linear, or tortuous port conduit pathway, and stream-line the insertion procedure.

Disclosed herein is a vascular access system for accessing a vasculature of a patient including, a subcutaneous port including a conduit and defining a non-linear path, and a needle formed of a first material, and including a flexible portion configured to deflect from an axis of the needle to traverse the non-linear path of the conduit.

In some embodiments, a distal portion of the needle can transition between a first configuration and a second configuration, in the first configuration an axis of the distal portion of the needle extends parallel to the axis of the needle, in the second configuration the axis of the distal portion of the needle extends at an angle relative to the axis of the needle. In some embodiments, the flexible portion includes a helical slit extending through a wall of the needle and extending in a spiral path about the axis of the needle. In some embodiments, the flexible portion includes a braided wire portion. In some embodiments, the flexible portion includes a first plurality of slits, each slit of the plurality of slits extending through a wall of the needle and extending perpendicular to the axis of the needle.

In some embodiments, a mid-point of each slit of the first plurality of slits are aligned with a first radial position about the axis of the needle, the first plurality of slits are configured to allow the needle to flex from a straight configuration to a deflected configuration along a first plane extending parallel to the axis of the needle. In some embodiments, a distal tip includes a bevel, a proximal-most edge of the bevel being aligned with the first radial position of the needle. In some embodiments, the vascular access system further includes a second plurality of slits, a mid-point of the second plurality of slits is aligned with a second radial position about the axis of the needle, and configured to allow the needle to flex from a straight configuration to a deflected configuration along a second plane, the second plane extending parallel to the axis of the needle and extending at angle relative to the first plane. In some embodiments, the angle of the second plane relative to the first plane is between 1° and 359°. In some embodiments, the angle of the second plane relative to the first plane is between 15° and 180°.

In some embodiments, the flexible portion includes a second material different from the first material, the second material providing more flexible mechanical characteristics relative to the first material. The second material is selected from a group consisting of a metal, alloy, nitinol, plastic, polymer, elastomer, and a composite. In some embodiments, the vascular access system further includes a needle guide configured to engage the port when the port is disposed subcutaneously, and align the needle with the conduit, the flexible portion configured to extend through a needle channel of the needle guide. In some embodiments, the vascular access system further includes a cannula disposed on an outer surface of the needle and slidably engaged therewith.

Also disclosed is a method of accessing a subcutaneous port including, advancing a distal tip of a needle through a conduit of the subcutaneous port, the conduit defining a non-linear path, deflecting a flexible portion of the needle from an axis of the needle to transition the distal tip from a straight configuration to a deflected configuration, and advancing the distal tip distally of the conduit.

In some embodiments, the method further includes withdrawing the needle from a lumen of a cannula once a distal tip of the cannula is disposed distally of the conduit, the cannula disposed on an outer surface of the needle.

In some embodiments, the method further includes advancing a distal tip of a cannula distally of a valve, the valve disposed at a distal end of the conduit.

In some embodiments, the flexible portion includes a helical slit extending through a wall of the needle and extending in a spiral path about the axis of the needle.

In some embodiments, the flexible portion includes a braided wire portion.

In some embodiments, the flexible portion includes a first plurality of slits, each slit of the plurality of slits extending through a wall of the needle and extending perpendicular to the axis of the needle.

In some embodiments, the method further includes deflecting the flexible portion along a first plane extending parallel to the axis of the needle, a mid-point of the first plurality of slits are aligned with a first radial position and aligned opposite from the first plane across the axis of the needle.

In some embodiments, the distal tip includes a bevel, a proximal-most edge of the bevel face is aligned with the first radial position.

In some embodiments, the method further includes a second plurality of slits, a mid-point of the second plurality of slits are aligned with a second radial position and configured to flex through a second plane, the second plane extending parallel to the axis of the needle and extending at angle relative to the first plane.

In some embodiments, the angle of the second plane relative to the first plane is between 1° and 359°.

In some embodiments, the angle of the second plane relative to the first plane is between 15° and 180°.

In some embodiments, the needle is formed of a first material and the flexible portion is formed of a second material different from the first material, the second material providing more flexible mechanical characteristics relative to the first material.

In some embodiments, the second material is selected from a group consisting of a metal, alloy, nitinol, plastic, polymer, elastomer, and a composite.

In some embodiments, the method further includes engaging a needle guide with the subcutaneous port, aligning the needle with the conduit, and advancing the flexible portion through a needle channel of the needle guide.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows a cross-section view of a subcutaneous port and rigid needle, in accordance with embodiments disclosed herein.

FIG. 2A shows a side view of a flexible needle in a straight configuration, in accordance with embodiments disclosed herein.

FIG. 2B shows a side view of a flexible needle in a deflected configuration, in accordance with embodiments disclosed herein.

FIG. 3 shows a side view of a flexible needle engaged with a subcutaneous port, the port shown in wire frame, in accordance with embodiments disclosed herein.

FIG. 4 shows an access assembly including a subcutaneous port, a flexible needle, and a needle guide, in accordance with embodiments disclosed herein.

FIG. 5A shows a side view of a flexible needle in a straight configuration and including a braided flexible portion, in accordance with embodiments disclosed herein.

FIG. 5B shows a side view of a flexible needle in a deflected configuration, the flexible portion including a second material, in accordance with embodiments disclosed herein.

FIG. 5C shows a side view of a flexible needle, the flexible portion including a plurality of slits, in accordance with embodiments disclosed herein.

FIG. 5D shows a cross-section view of the flexible needle of FIG. 5C, in accordance with embodiments disclosed herein.

FIG. 6A shows a side view of a flexible needle, the flexible portion including a first plurality of slits, in accordance with embodiments disclosed herein.

FIG. 6B shows a cross-section view of the flexible needle of FIG. 6A, in accordance with embodiments disclosed herein.

FIG. 6C shows a side view of a flexible needle, the flexible portion including a first plurality of slits and a second plurality of slits, in accordance with embodiments disclosed herein.

FIG. 6D shows a cross-section view of the flexible needle of FIG. 6C, in accordance with embodiments disclosed herein.

FIGS. 7A-7B shows a side view of a flexible needle engaged with a subcutaneous port, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a needle disclosed herein includes a portion of the needle intended to be further from a patient, when the needle is used on the patient. Likewise, a “proximal length” of, for example, the needle includes a length of the needle intended to be further from the patient when the needle is used on the patient. A “proximal end” of, for example, the needle includes an end of the needle intended to be further from the patient when the needle is used on the patient. The proximal portion, the proximal end portion, or the proximal length of the needle can include the proximal end of the needle; however, the proximal portion, the proximal end portion, or the proximal length of the needle need not include the proximal end of the needle. That is, unless context suggests otherwise, the proximal portion, the proximal end portion, or the proximal length of the needle is not a terminal portion or terminal length of the needle.

With respect to “distal,” a “distal portion” or a “distal end portion” of, for example, a needle disclosed herein includes a portion of the needle intended to be near or in a patient when the needle is used on the patient. Likewise, a “distal length” of, for example, the needle includes a length of the needle intended to be near or in the patient when the needle is used on the patient. A “distal end” of, for example, the needle includes an end of the needle intended to be near or in the patient when the needle is used on the patient. The distal portion, the distal end portion, or the distal length of the needle can include the distal end of the needle; however, the distal portion, the distal end portion, or the distal length of the needle need not include the distal end of the needle. That is, unless context suggests otherwise, the distal portion, the distal end portion, or the distal length of the needle is not a terminal portion or terminal length of the needle.

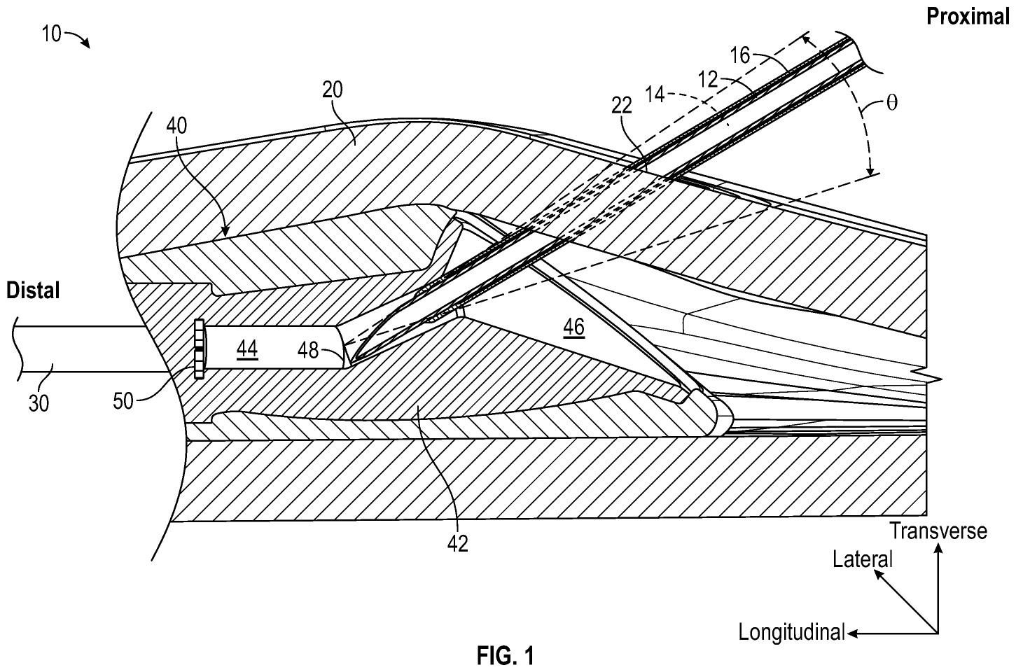

To assist in the description of embodiments described herein, as shown in FIG. 1, a longitudinal axis extends substantially parallel to an axial length of the port 40 and catheter 30. A lateral axis extends normal to the longitudinal axis, and a transverse axis extends normal to both the longitudinal and lateral axes.

Unless defined otherwise, all technical and scientific terms used herein have the same meaning as commonly understood by those of ordinary skill in the art.

FIG. 1 shows an exemplary access system 10 including a rigid needle 12, with a soft cannula 16 disposed thereon, accessing a subcutaneous access port 40. The rigid needle 12 can be formed of stainless steel, or similar suitable material and optionally can define a needle lumen 14. In an embodiment, the cannula 16 can be a peripherally inserted venous (PIV) catheter, or similar suitable device, slidably engaged with the rigid needle 12. However, the PIV catheter is exemplary and not intended to be limiting in any way. The cannula 16 can be formed of a soft, compliant material such as a plastic, polymer, latex, rubber, silicone rubber, or the like.

The port 40 can be a subcutaneous access port, or similar subcutaneous medical device configured to provide fluid communication with a vasculature of a patient. However, it will be appreciated that other subcutaneous, needle-accessible, medical devices are also contemplated to fall within the scope of the present invention. The port 40 can be in fluid communication with a catheter 30, a distal tip of the catheter 30 can be disposed within a vasculature of a patient and provide fluid communication therewith.

In an embodiment, the port 40 can generally include a port body 42 defining a conduit 44 that is in fluid communication with a lumen of a catheter 30 at a distal end thereof. A proximal end of the conduit 44 can define a funnel shaped, receiving cup 46 defining a tapered profile and configured to direct a needle, e.g. a rigid needle 12 inserted therein, into the conduit 44. In an embodiment, the port 40 can include a needle penetrable septum, a valve, or the like, configure to control a fluid flow through the port 40. Optionally, the port 40 can include a reservoir in fluid communication with the conduit 44. The port 40 can define a transverse height and can define an outer profile configured to be palpated by a user to locate the port 40 when disposed subcutaneously. In an embodiment, the port 40 can define a distinctive outer profile to facilitate orientation of the port 40 and location of the receiving cup 46 for a user to insert a needle therein.

As shown in FIG. 1, the receiving cup 46 can define a relatively wide angle configured to capture a distal tip of the rigid needle 12 and direct the rigid needle 12 into the conduit 44. As will be appreciated, however, lateral and/or transverse movement of the rigid needle 12 can be restricted by interaction between the needle 12 and the skin 20 at the insertion site 22. Lateral or transverse movement of the rigid needle 12 is further restricted by the width of the conduit 44. As such, a suitable “angle of attack” (“θ”) is required to access the port 40, wherein a rigid needle 12 can align with the conduit 44 and provide a successful fluid connection therewith. As will be appreciated, when accessing the port 40 with a rigid needle 12, the angle of attack (“θ”) can be relatively narrow, leading to an increased risk of mis-sticks.

In addition, assuming the rigid needle 12 is inserted through the skin 20 and aligned correctly with the conduit 44, the rigid needle 12 can only be advanced into a straight portion of the conduit 44. Any further advancement can be inhibited by a first inflection point 48 in the conduit 44 pathway. Once the rigid needle 12 has impinged on the wall of the conduit 44, e.g. at the inflection point 48, the user must retract, or “back off” the needle 12 a short distance, but not too far so as to remove the needle tip from the port conduit 44 altogether. The soft cannula 16 can then be advanced over the rigid needle 12 and into the port conduit 44, past the first inflection point 48 and optionally, passing through any valve elements 50.

If the rigid needle 12 is not exactly aligned with the conduit 44, the needle tip can impinge against a portion of the conduit wall proximal to the inflection point 48, increasing the risk of buckling or kinking of the cannula 16. Should the rigid needle 12 be incorrectly aligned with the port 40, the rigid needle 12 can even impinge on a wall of the receiving cup 46 and fail to access the conduit 44 altogether.

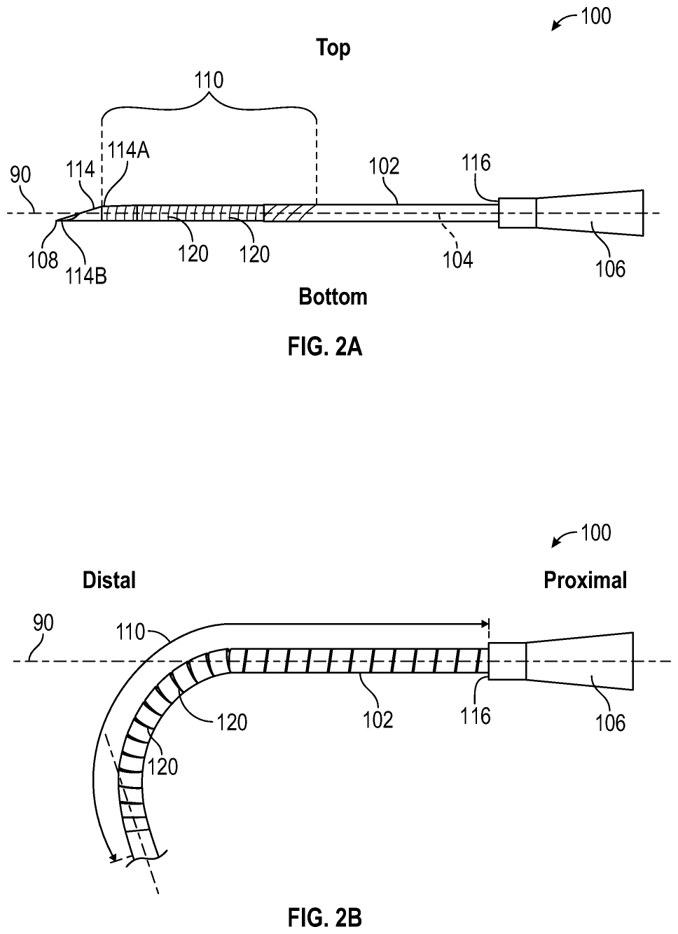

FIGS. 2A-2B show an embodiment of a flexible needle 100 configured to access a port 40 and advance through the non-linear, or tortuous, pathway of the port conduit 44. The flexible needle (“needle”) 100 can generally include an elongate body 102 extending along an axis 90 and defining a lumen 104. The elongate body 102 can include a sharpened distal tip 108 and can be supported at a proximal end by a hub 106. The sharpened distal tip 108 can define a bevel face 114 angled relative to a central axis 90 of the needle 100. The hub 106 can facilitate manipulation of the needle 100 and, in an embodiment, provide fluid communication with the lumen 104. Optionally, the hub 106 can include a connector, luer lock, or the like, configured to facilitate providing fluid communication with the lumen 104 of the needle 100. In an embodiment, the elongate body 102 can define a tubular structure formed of metal, stainless steel, alloy, plastic, polymer, composite, or similar suitable material. In an embodiment, the bevel face 114 can be angled “upwards” towards a top surface of the needle 100 such that a proximal-most edge 114A of the bevel face 114 is disposed top-most, and a distal-most edge 114B of the bevel face 114 is disposed bottom-most. It will be appreciated, however, that other orientations of the bevel face 114 are also contemplated.

In an embodiment, the needle 100 can further include a flexible portion 110 extending along at least a portion of the tubular body 102. The flexible portion 110 can be configured to allow a portion of the body 102 to deflect from the central axis 90 of the needle 100. For example, as shown in FIG. 2B, a flexible portion 110 can allow a distal portion, e.g. the distal tip 108, of the needle 100 to deflect from the axis 90 of the needle 100. In an embodiment, the flexible portion 110 can extend along substantially the entire axial length of the tubular body 102.

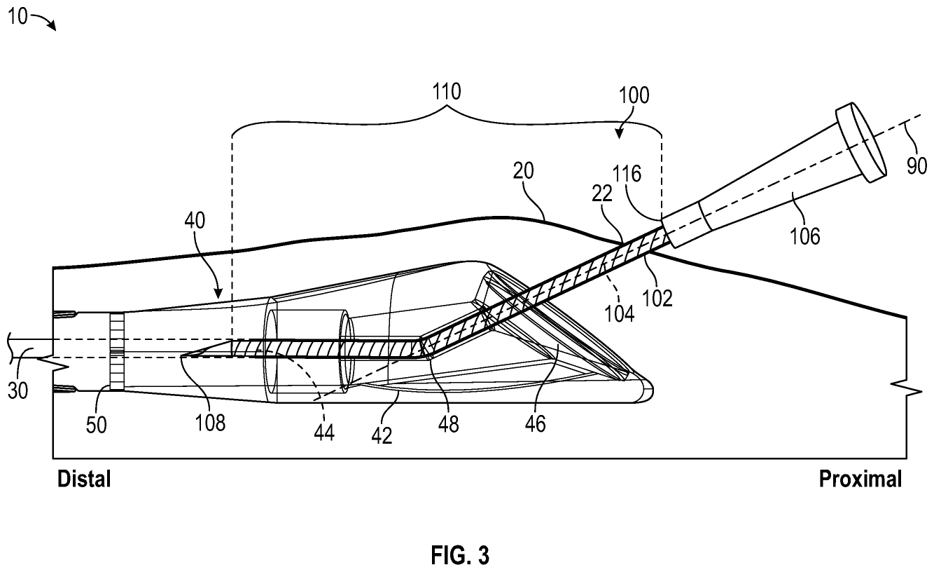

FIG. 3 shows an embodiment of an access assembly 10 including a subcutaneous port 40 in fluid communication with a catheter 30, and a flexible needle 100 engaged with the port 40. In an embodiment, the flexible portion 110 of the needle 100 can allow the distal tip 108 of the needle 100 to extend through a non-linear conduit 44 of the port 40. Advantageously, the flexible needle 100 can allow a portion of the needle 100 to deflect from the central axis 90 of the needle 100. Further, the flexible portion 110 can maintain the columnar strength of the needle 100 when an axial compressive force is applied thereto. As such, the needle 100 can be urged distally to pierce the skin 20 and a distal tip 108 can be urged through the conduit 44 to access the port 40 without the needle 100 buckling or kinking.

In an embodiment, once engaged with the port 40, the needle lumen 104 can provide a fluid pathway with the catheter 30 and provide fluid communication therewith. In an embodiment, the needle 100 can provide mechanical support to a soft cannula 16, disposed on an outer surface of the needle body 102, as the needle 100 and cannula 16 assembly is urged through the conduit 44 of the port 40. In an embodiment, the needle 100 can provide mechanical support to the cannula 16 as a distal tip of the cannula 16 is urged distally through the conduit 44, and optionally past a valve structure 50. Once the cannula 16 is engaged with the port 40, the needle 100 can be withdrawn proximally, leaving the cannula 16 in place to provide fluid communication with the port 40 and catheter 30.

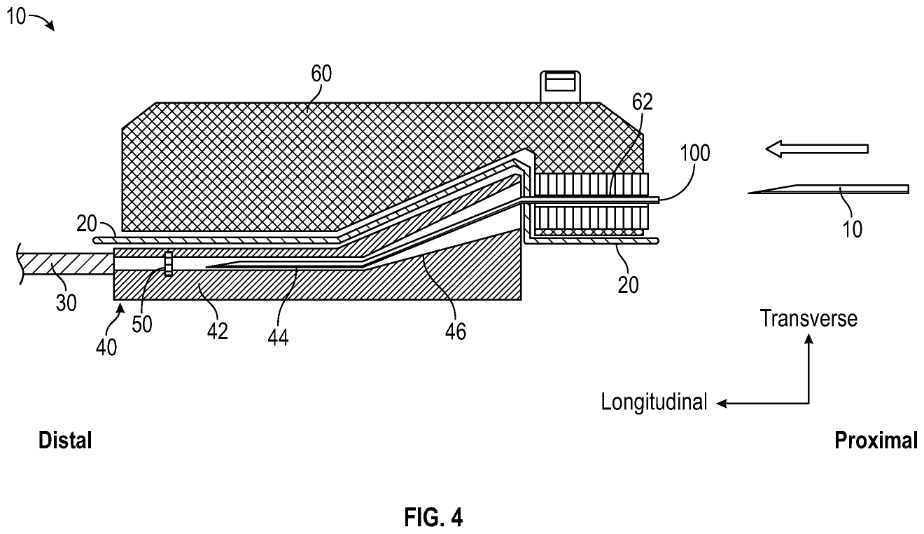

FIG. 4 shows an embodiment of an access assembly 10 including a subcutaneous port 40 in fluid communication with a catheter 30, a needle guide 60 engaged with a skin surface 20, and a flexible needle 100 engaged with the port 40/needle guide assembly. In an embodiment, the needle guide 60 can be configured to engage a skin surface 20 and the port 40, disposed subcutaneously, and align the flexible needle 100 with the conduit 44 of the port 40.

In an embodiment, the needle guide 60 can be configured to engage the port 40 and align a needle channel 62 with the receiving cup 46 of the port 40. A user can then insert the flexible needle 100 through the needle channel 62 of the needle guide 60, pierce the skin 20 and insert the flexible needle 100 into the receiving cup 46 and the conduit 44 of the port 40. As will be appreciated, the addition of the needle guide 60 can increase the tortuous pathway that a needle 100 must negotiate before fully engaging the port 40. As such, a rigid needle 12 may not be able to access the port 40 fully, whereas embodiments of the flexible needle 100 described herein can negotiate the tortuous pathway of the needle channel 62 and the conduit 44, while mitigating buckling or kinking.

In an embodiment, as shown in FIGS. 2A-2B, the flexible needle 100 can include an abutment 116 configured to engage one or more of a skin surface, a portion of the port 40, or a portion of a needle guide 60, and configured to prevent the flexible needle 100 extending further into the port 40. In an embodiment, a distance between the abutment 116 and the tip 108 of the needle 100 can be less than a distance between a valve structure 50 and one of the skin surface, the portion of the port 40, or the portion of a needle guide 60. Advantageously, the abutment 116 can prevent the needle tip 108 from engaging the valve structure 50 and causing any damage thereto.

In an embodiment, as shown in FIGS. 2A-2B, the flexible portion 110 can include a helical slit 120 extending through a wall of the needle 100 between an outer surface of the needle 100 and the needle lumen 104. The helical slit 120 can also extend about the axis 90 of the flexible needle 100 to define a helical shape. The helical slit 120 can allow adjacent portions of the needle wall to slide relative to each other and allow the needle 100 to flex, and deflect from the needle axis 90 when a force is applied to the needle 100 at an angle relative to the needle axis 90. Further, when an axial force is applied to the needle 100, e.g. either a proximal or distal compressive force, the adjacent portions of the needle wall of the flexible portion 110 can abut against each other and can maintain columnar strength, mitigating buckling or kinking. Worded differently, the flexible portion 110 can include a ribbon of material that is wound around a central axis 90 of the flexible needle 100 in a spiral, or helical shape, to form a portion of the lumen 104.

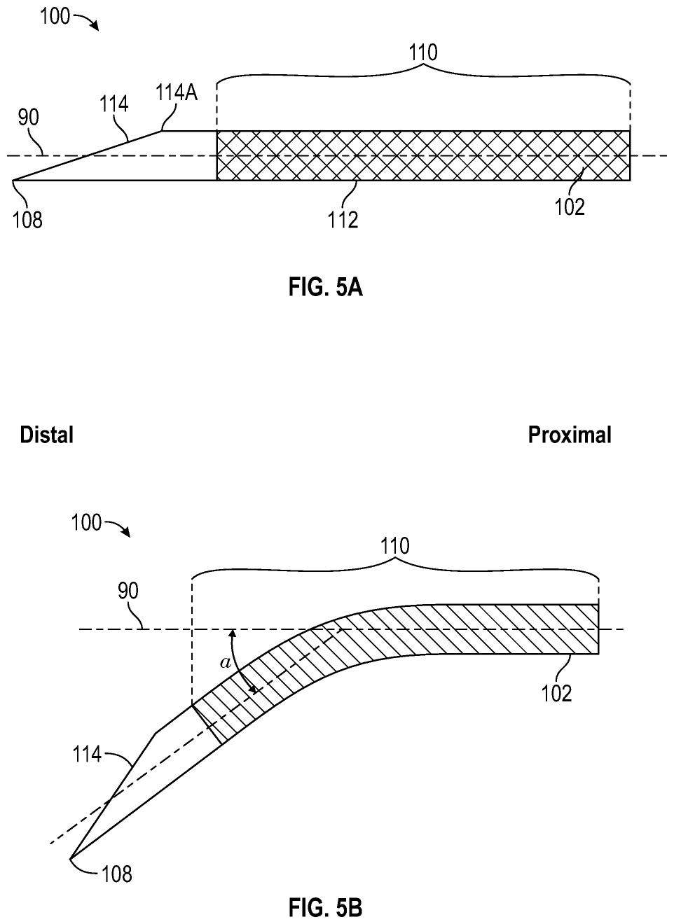

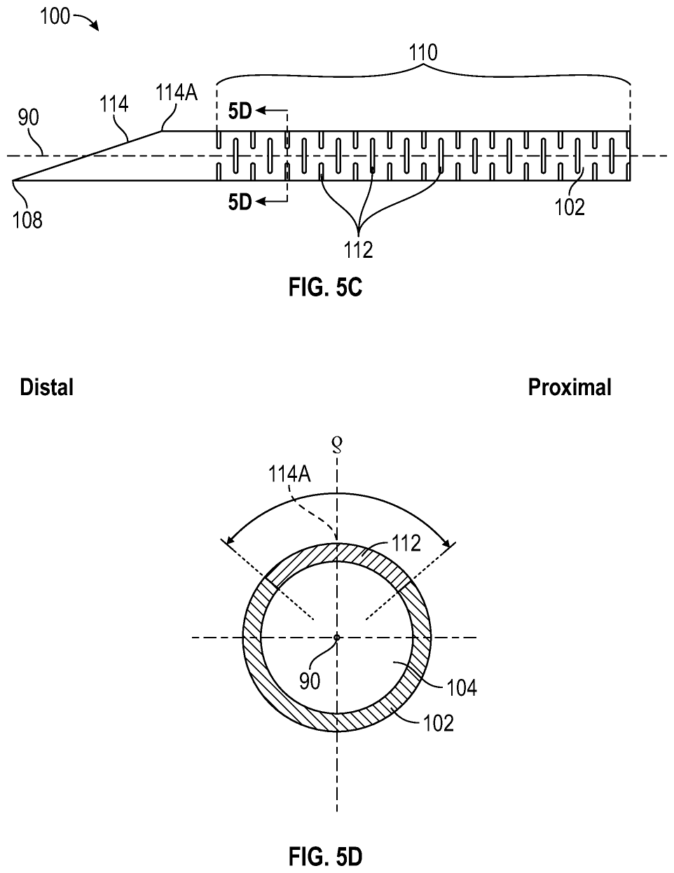

In an embodiment, as shown in FIG. 5A, the flexible portion 110 can include a braided portion including a plurality of wires interwoven and encircling the central axis 90. The braided flexible portion 110 can form a portion of the wall of the needle and define a portion of the needle lumen 104 therethrough. The braided flexible portion 110 can be configured to allow the needle 100 to flex, and deflect from the needle axis 90 when a force is applied to the needle 100 at an angle relative to the needle axis 90. Further, when an axial force is applied to the needle 100, e.g. either a proximal or distal compressive force, the wires of the braided flexible portion 110 can interlock against each other and can maintain columnar strength. In an embodiment, the flexible portion 110 can be configured to allow the needle 100 to flex and deflect from the needle axis 90 along a single plane or along multiple planes, as described in more detail herein.

In an embodiment, as shown in FIG. 5B, the tubular body 102 can be formed of a first material and the flexible section 110 can include a second material, different from the first material. In an embodiment, the first material can be a metal, stainless steel, titanium, alloy, plastic, polymer, or the like. In an embodiment, the second material can be a metal, alloy, nitinol, plastic, polymer, elastomer, or the like. In an embodiment, the first material can include rigid mechanical properties and the second material can include more flexible mechanical properties, relative to the first material. In an embodiment, the second material can include elastically deformable mechanical properties. In an embodiment, the second material can include plastically deformable mechanical properties, also termed “malleable.” In an embodiment, the second material can extend through a portion of the flexible portion 110. In an embodiment, the flexible portion 110 can be formed entirely of the second material.

In an embodiment, the flexible portion 110 including the second material can be configured to allow the needle 100 to flex, and deflect from the needle axis 90 when a force is applied to the needle 100 at an angle relative to the needle axis 90. Further, when an axial force is applied to the needle 100, e.g. either a proximal or distal compressive force, the flexible portion 110 including the second material can maintain columnar strength, as described herein.

In an embodiment, as shown in FIG. 5C, the flexible portion 110 can include a plurality of slits 112 extending from an outer surface of the wall of the needle 100 to a lumen 104 of the needle 100. As used herein, the term “slit” can include an aperture where opposing walls are in contact with each other when in a relaxed state, but can deform to a stressed state where at least a portion of the opposing walls are in a spaced apart relationship. In an embodiment, the plurality of slits 112 can include one or more apertures extending between an outer surface of the needle 100 and the lumen 104. As used herein, the term “aperture” can include an opening where opposing walls of the opening are in a spaced apart relationship when in a relaxed state. The opposing walls of the aperture can elastically deform to either contact each other, to a relatively closer together position, or a relatively further spaced apart relationship in a stressed, or elastically deformed, state. The aperture can be positioned similarly to a slit of the plurality of slits 112, as described herein. In an embodiment, the aperture can define an oblong, circular, elliptical shape, or similar regular or irregular closed curve polygon. In an embodiment, the plurality of slits 112 or apertures can be formed by laser cut, punch out, or by similar suitable means.

In an embodiment, a slit of the plurality of slits 112 can extend at an angle relative to the central axis 90 of the needle. For example, as shown in FIG. 5C, each slit of the plurality of slits can extend perpendicular to the axis 90 of the needle 100. However, it will be appreciated that each slit of the plurality of slits 112 can extend at an angle of between 0° and 180° relative to the central axis 90. In an embodiment, each slit of the plurality of slits 112 can extend at the same angle relative to each other, or at different angles relative to each other.

As shown in FIG. 5D, in an embodiment, each slit of the plurality of slits 112 can extend about the central axis 90 by an arc distance (g) of between 1° and 355°. In an embodiment, each slit of the plurality of slits 112 can extend about the central axis 90 by an arc distance (g) of between 15° and 150°. In an embodiment, a mid-point of each slit of the plurality of slits 112 can be disposed the same, or at different radial positions about the central axis 90. For example, as shown in FIG. 5D, the mid-point of a slit of the plurality of slits 112 is disposed at the “12 o'clock” radial position, or along the top side of the needle, substantially in line with a proximal-most edge 114A. However, it will be appreciated that the mid-point of the slit of the plurality of slits 112 can be position at any radial position about the central axis 90. In an embodiment, the slits of the plurality of slits 112 can be disposed in linear, spiral, or alternating pattern relative to each other, about the outer surface of the tubular body 102. However, it will be appreciated that other configurations of the plurality of slits 112 are also contemplated. Advantageously, the plurality of slits 112 and/or apertures can allow the flexible portion 110 to be sufficient flexible to allow a distal portion of the needle 100 to deflect from the central axis 90, while maintaining sufficient columnar strength to mitigate kinking or collapsing.

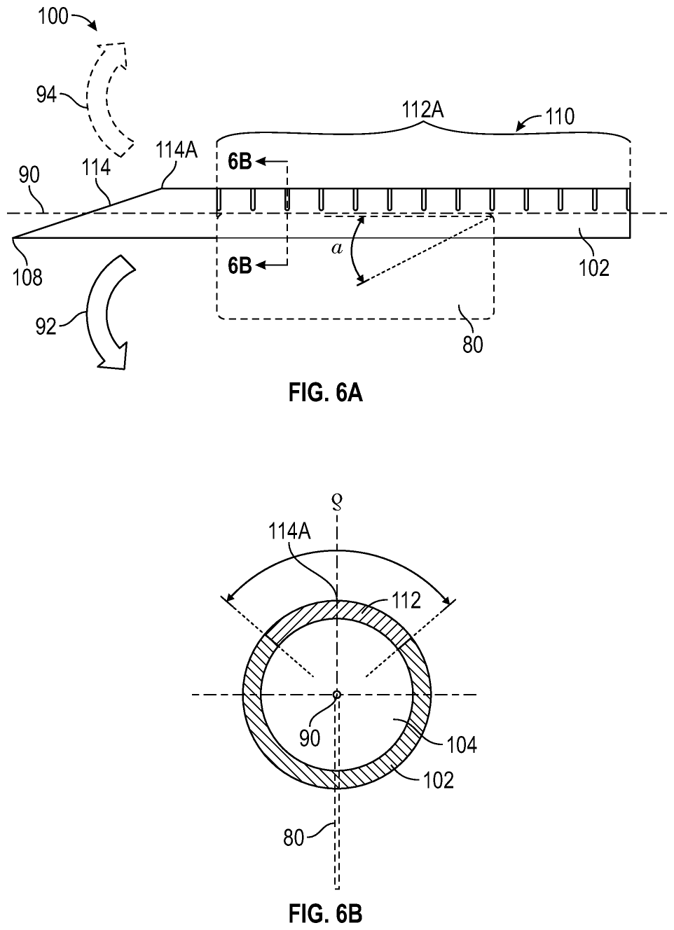

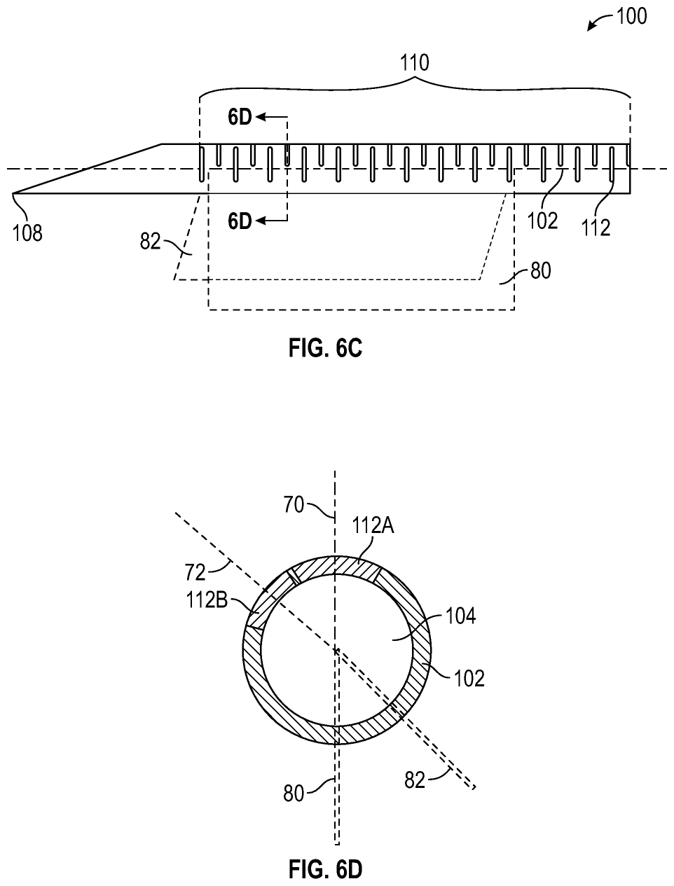

In an embodiment, as shown in FIG. 6A, the flexible portion 110 can include a first plurality of slits 112A. A mid-point of each slit of the first plurality of slits 112A can be aligned with the central axis 90, at a first radial position, for example, along a top side of the needle body 102. Each slit of the first plurality of slits 112A can extend through a wall of the needle by an arc distance (p). As such, the first plurality of slits 112A can be configured to allow the needle 100 to flex in a first direction 92 through a first plane 80. The first plane 80 can be aligned with the central axis 90 and extend perpendicular from the central axis 90, away from the first plurality of slits 112A.

Advantageously, the first plurality of slits 112A can allow the flexible portion 110 to flex through a first plane 80, e.g. a longitudinal vertical plane, in a first direction 92, while restricting movement through other planes or other directions, e.g. through the longitudinal vertical plane in a second direction 94 opposite the first direction, a longitudinal horizontal plane, or other planes extending at an angle relative to the first plane 80, as described in more detail herein. In an embodiment, the first plurality of slits 112A can allow the flexible portion 110 to flex through the first plane 80 by an angle (“α”). In an embodiment, the angle (“α”) can be between 1° and 180° relative to the central axis 90. In an embodiment, the angle (“α”) can be between 15° and 80° relative to the central axis 90.

In an embodiment, as shown in FIGS. 6C-6D, the flexible portion 110 can include a first plurality of slits 112A aligned with a first radial position 70, as described herein, and a second plurality of slits 112B wherein a mid-point of each slit of the second plurality of slits 112B can be aligned with a second radially position 72. As such, the first plurality of slits 112A can allow the flexible portion 110 to flex through a first plane 80, and the second plurality of slits 112B can allow the flexible portion 110 to flex through a second plane 82 disposed at an angle relative to the first plane 80.

In an embodiment, the slits of the first plurality of slits 112A can be interposed between the slits of the second plurality of slits 112B. In an embodiment the slits of the first plurality of slits 112A can be disposed alternately with the slits of the second plurality of slits 112B. In an embodiment, the slits of the first plurality of slits 112A can be disposed towards a first end of the flexible portion 110, e.g. a proximal end, and the slits of the second plurality of slits 112B can be disposed towards a second end of the flexible portion 110, e.g. a distal end. However, it will be appreciated that these and other combinations, numbers, and orientations of plurality of slits are also contemplated. In an embodiment, the second radial position 72 can be disposed at an angle relative to the first radial position 70 of between 1° and 359°. In an embodiment, the second radial position 72 can be disposed at an angle relative to the first radial position 70 of between 5° and 90°.

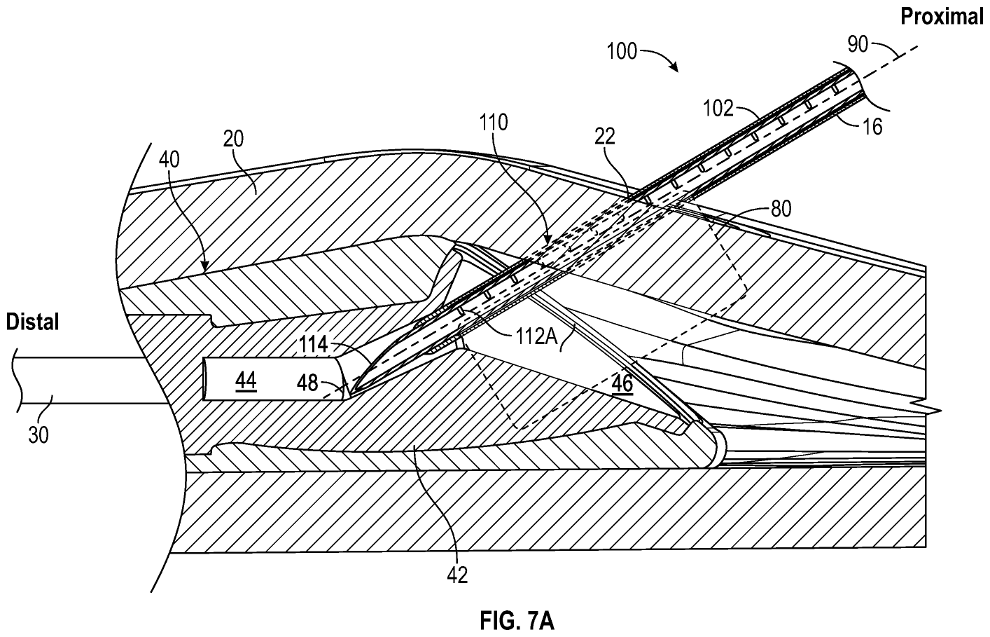

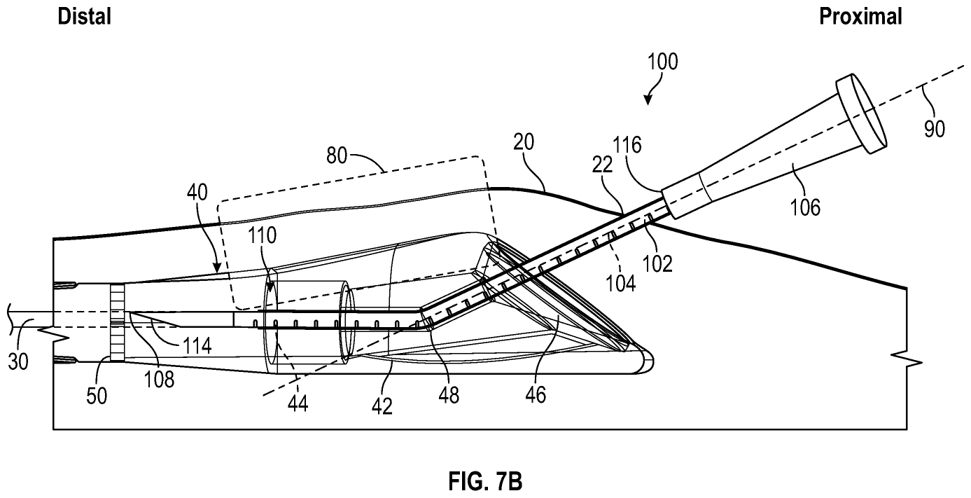

In an embodiment, the flexible portion 110 can be configured to flex through one or more predetermined planes, e.g. one of the first plane 80 or the second plane 82 while restricting movement through other planes. For example, as shown in FIGS. 7A-7B, the needle 100 can include the flexible portion 110 having the first plurality of slits 112A, as described herein. As such, the flexible portion 110 can be restricted to flex through only the first plane 80, i.e. away from the top side of the needle 100. As the user inserts the needle 100 with the bevel face 114 facing upwards, the needle 100 can be advanced up to the first inflection point 48. Further advancement of the distal tip 108 is restricted since the inflection point 48 is oriented in the opposite direction from first plane 80.

As such, as shown in FIG. 7B the user can rotate the flexible needle 100 about the central axis 90, until the bevel face 114 is facing downwards. The first plane 80 is then aligned with the direction of the first inflection point 48 and the flexible portion 110 can flex to allow the needle 100 to traverse the first inflection point 48. Advantageously, the flexible portion 110 can be configured as such so that the sharpened distal tip 108 is rotated away from the first inflection point 48, preventing the sharpened distal tip 108 from impinging on a wall of the conduit, blunting the tip 108 or damaging the port 40. The flexible portion 110 can be configured to direct a user to insert the needle 100 into the port 40 while mitigating damage to the port 40 that might be caused by the sharpened needle tip 108.

It is important to note, while the flexible portion 110 with the plurality of slits 112A has been used as an example of how the flexible portion 110 can be configured to flex in a particular direction or plane. It will be appreciated, however, that this is a simplified example and the flexible portion 110 can include two or more plurality of slits 112 configured to allow the needle 100 to flex in one or more predetermined planes or directions.

Further, it will be appreciated that other configurations of flexible portion 110, including a spiral slit 120, braided portion, or one or more materials displaying one or more mechanical characteristics can also be configured to restrict flexion of the flexible portion 110 to one or more particular planes or directions from the central axis 90. For example, the flexible portion 110 can include a first material extending along a bottom side of the needle, and a second material extending along a top side of the needle, substantially in line with the proximal-most edge 114A of the bevel face 114. As such, the second material can allow the flexible portion to elastically deform, allowing the needle to flex in a first direction through the first plane 80. These and other combinations are considered to fall within the scope of the present invention.

In an exemplary method of use, an access system 10 is provided including a port 40 and a flexible needle 100, as described herein. In an embodiment, the access system 10 can further include a needle guide 60 configured to engage the port 40 and align the needle 100 with a conduit 44 of the port 40. In an embodiment, the needle 100 can further include a soft cannula 16 slidably engaged with an outer surface of the flexible needle 100. In an embodiment, the flexible needle 100 can further include a flexible portion 110 configured to allow the distal tip 108 of the flexible needle 100 to deflect from a central axis 90 of the flexible needle 100. In an embodiment, the flexible portion 110 can be configured to allow the distal tip 108 to deflect along one of a first plane 80 or in a first direction along the first plane 80.

A user can advance the needle 100 distally to pierce the skin 20 at the insertion site 22 and urge the distal tip 108 into the conduit 44 of the port 40. In an embodiment, should the insertion angle of the flexible needle 110 be mis-aligned with the axis of the conduit 44, the flexible portion 110 can allow the distal tip 108 to impinge on the receiving cup 46. The receiving cup 46 can then direct the needle tip 108 towards the entrance of the conduit 44.

Once the distal tip 108 is engaged with the conduit 44, the needle 100 can be advanced distally through the conduit 44. In an embodiment, the pathway of the conduit 44 can be non-linear and include one or more inflection points, e.g. first inflection point 48. The flexible portion 110 can be configured to allow the distal tip 108 to be advanced distally past the first inflection point 48.

In an embodiment, the flexible portion 110 can be configured to restrict deflection of the distal tip 108 to a first plane 80 and/or a first direction 92. As such, a user can rotate the needle 100 about the central axis 90, for example, until a bevel face 114 is directed towards the inflection point 48 and the sharpened distal tip 108 is rotated away from the inflection point 48. The flexible portion 110 can then allow the distal tip 108 to be advanced distally of the first inflection point 48. As such, the flexible portion 110 can be configured to mitigate impingement of the sharpened distal tip 108 against the wall of the conduit 44 as the needle tip 108 is advanced distally of the first inflection point 48.

As will be appreciated, where the access system 10 includes a needle guide 60, a needle channel 62 may increase the tortuous path, or the number of inflection points that the needle 100 must negotiate before fully engaging the port 40. In an embodiment, the flexible 100 can be advanced distally until fully engaged with the port conduit 44. The lumen 104 of the needle 100 can then provide fluid communication with the port conduit 44 and the catheter 30. In an embodiment, the needle 100 including the cannula 16 disposed thereon can be advanced until the cannula 16 is fully engaged with the port 40. The needle 100 can then be withdrawn proximally, leaving the cannula 16 disposed in place to provide fluid communication with the conduit 44 and catheter 30. In an embodiment, the needle 100 and optionally the cannula 16 can be advanced distally of the valve structure 50. Advantageously, in an embodiment, the needle lumen 104 can define a fluid pathway through the valve 50 to provide fluid communication with the catheter 30. In an embodiment, the needle 100 can provide columnar support to the cannula 16 as a portion of the needle 100 and cannula 16 assembly is urged distally through the valve structure 50. The needle 100 can then be withdrawn proximally and the cannula 16 can provide a fluid pathway through the valve structure 50 to the catheter 30.

In an embodiment, the needle 100 can include an abutment 116 configured to engage one of a skin surface, a portion of the port 40, or a portion of a needle guide 60, and configured to prevent the flexible needle 100 extending longitudinally further into the port 40. The abutment 116 can prevent the needle tip 108 from engaging the valve structure 50 mitigating any damage to the valve 50, while still providing columnar support to the cannula 16 up to the valve structure 50 mitigating kinking of buckling of the cannula 16 as the cannula 16 is slid distally off of the needle 100 and through the valve structure 50.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A vascular access system for accessing a vasculature of a patient, comprising:
a subcutaneous port including a conduit defining a non-linear path; and
a needle including a flexible portion configured to deflect from an axis of the needle to traverse the non-linear path of the conduit.

2. The vascular access system according to claim 1, wherein a distal portion of the needle can transition between a first configuration and a second configuration, in the first configuration an axis of the distal portion of the needle extends parallel to the axis of the needle, in the second configuration the axis of the distal portion of the needle extends at an angle relative to the axis of the needle.

3. The vascular access system according to claim 1, wherein the flexible portion includes a helical slit extending through a wall of the needle and extending in a spiral path about the axis of the needle.

4. The vascular access system according to claim 1, wherein the flexible portion includes a first plurality of slits, each slit of the plurality of slits extending through a wall of the needle and extending perpendicular to the axis of the needle.

5. The vascular access system according to claim 4, wherein a mid-point of each slit of the first plurality of slits are aligned with a first radial position about the axis of the needle, the first plurality of slits are configured to allow the needle to flex from a straight configuration to a deflected configuration along a first plane extending parallel to the axis of the needle.

6. The vascular access system according to claim 5, wherein a distal tip includes a bevel, a proximal-most edge of the bevel being aligned with the first radial position of the needle.

7. The vascular access system claim 4, further including a second plurality of slits, a mid-point of the second plurality of slits is aligned with a second radial position about the axis of the needle, and configured to allow the needle to flex from a straight configuration to a deflected configuration along a second plane, the second plane extending parallel to the axis of the needle and extending at angle relative to the first plane.

8. The vascular access system according to claim 7, wherein the angle of the second plane relative to the first plane is between 1° and 359°.

9. The vascular access system according to claim 7, wherein the angle of the second plane relative to the first plane is between 15° and 180°.

10. The vascular access system according to claim 1, wherein the needle is formed of a material selected from a group consisting of a metal, alloy, nitinol, plastic, polymer, and a composite.

11. The vascular access system according to claim 1, further including a needle guide configured to engage the port when the port is disposed subcutaneously, and align the needle with the conduit, the flexible portion configured to extend through a needle channel of the needle guide.

12. The vascular access system according to claim 1, further including a cannula disposed on an outer surface of the needle and slidably engaged therewith.

13. A method of accessing a subcutaneous port, comprising:
advancing a distal tip of a needle through a conduit of the subcutaneous port, the conduit defining a non-linear path;
deflecting a flexible portion of the needle from an axis of the needle to transition the distal tip from a straight configuration to a deflected configuration; and
advancing the distal tip distally of the conduit.

14. The method according to claim 13, further including withdrawing the needle from a lumen of a cannula once a distal tip of the cannula is disposed distally of the conduit, the cannula disposed on an outer surface of the needle.

15. The method according to claim 14, further including advancing a distal tip of a cannula distally of a valve, the valve disposed at a distal end of the conduit.

16. The method according to claim 13, wherein the flexible portion includes a helical slit extending through a wall of the needle and extending in a spiral path about the axis of the needle.

17. The method according to claim 13, wherein the flexible portion includes a first plurality of slits, each slit of the plurality of slits extending through a wall of the needle and extending perpendicular to the axis of the needle.

18. The method according to claim 17, further including deflecting the flexible portion along a first plane extending parallel to the axis of the needle, a mid-point of the first plurality of slits are aligned with a first radial position and aligned opposite from the first plane across the axis of the needle.

19. The method according to claim 18, wherein the distal tip includes a bevel, a proximal-most edge of the bevel face is aligned with the first radial position.

20. The method according to claim 17, further including a second plurality of slits, a mid-point of the second plurality of slits are aligned with a second radial position and configured to flex through a second plane, the second plane extending parallel to the axis of the needle and extending at angle relative to the first plane.

21. The method according to claim 20, wherein the angle of the second plane relative to the first plane is between 1° and 359°.

22. The method according to claim 20, wherein the angle of the second plane relative to the first plane is between 15° and 180°.

23. The method according to claim 13, wherein the needle is formed of a material including one of a metal, alloy, nitinol, plastic, polymer, or a composite.

24. The method according to claim 13, further including engaging a needle guide with the subcutaneous port, aligning the needle with the conduit, and advancing the flexible portion through a needle channel of the needle guide.
